(19) United States
(12) Patent Application Publication
(10) Pub. No.: US 2018/0177567 A1
(43) Pub. Date: Jun. 28, 2018

(54) DRILL GUIDE

(71) Applicant: PALTOP ADVANCED DENTAL SOLUTIONS LTD., Caesarea (IL)

(72) Inventors: Michael Klein, Maale Adumim (IL); Mordechai Mor Miles, Pardesiya (IL); Shlomo Hillel, Petach Tiqva (IL)

(73) Assignee: PALTOP ADVANCED DENTAL SOLUTIONS LTD., Caesarea (IL)

(21) Appl. No.: 15/845,103

(22) Filed: Dec. 18, 2017

Related U.S. Application Data
(60) Provisional application No. 62/438,031, filed on Dec. 22, 2016.

(51) Int. Cl. A61C 1/08; A61C 8/00

(52) U.S. Cl. CPC A61C 1/084; A61B 17/1739; A61C 8/0089

(57) ABSTRACT

Apparatus is provided for use with a dental handpiece, a drill bit, an oral surgical guide, and a bushing disposed in the oral surgical guide. The apparatus includes a rigid guide-sleeve having a proximal end and a distal end. The proximal end of the guide-sleeve is removably couplable to a distal end of the dental handpiece, such that a distal outer surface of the guide-sleeve and a drill bit coupled to the distal end of the dental handpiece are coaxial along a central longitudinal axis of the drill bit. The distal end of the guide-sleeve is sized and shaped to be slidably couplable with the bushing, such that upon the guide-sleeve being advanced into the bushing the dental handpiece is forced to align the central longitudinal axis of the drill bit to be coaxial with a central longitudinal axis of the bushing.

### CROSS REFERENCE TO RELATED APPLICATIONS

The present application claims the priority of U.S. 62/438,031 to Klein, filed Dec. 22, 2016 entitled, “Drill Guide,” which is incorporated herein by reference.

### FIELD OF THE INVENTION

The present invention relates to centering and guiding a dental drill bit for drilling into a patient's bone.

### SUMMARY OF THE INVENTION

A rigid guide-sleeve is provided for use with a dental handpiece, a drill bit, and a bushing disposed in an oral surgical guide. The guide-sleeve is sized and shaped to slide into the bushing, and is couplable to the distal end of the dental handpiece such that the guide-sleeve and drill bit are coaxial along the central longitudinal axis of the drill bit. Upon the guide-sleeve being advanced into the bushing, the dental handpiece is forced to align the central longitudinal axis of the drill bit to be coaxial with the central longitudinal axis of the bushing.

In addition, a method is described for placing the distal tip of the drill bit into the bushing at an angle and then reorienting the guide-sleeve to align with and slide into the bushing. The reorientation of the guide-sleeve forces the dental handpiece to reorient, which in turns reorients the drill bit. The reoriented bit can then be advanced into the tissue of the patient.

There is therefore provided, in accordance with some applications of the present invention, apparatus for use with a dental handpiece, a drill bit, an oral surgical guide, and a bushing disposed in the oral surgical guide, the apparatus including:

a rigid guide-sleeve having a proximal end and a distal end,
the proximal end of the guide-sleeve being removably couplable to a distal end of the dental handpiece, such that a distal outer surface of the guide-sleeve and a drill bit coupled to the distal end of the dental handpiece are coaxial along a central longitudinal axis of the drill bit, and
the distal end of the guide-sleeve being sized and shaped to be slidably couplable with the bushing, such that upon the guide-sleeve being advanced into the bushing the dental handpiece is forced to align the central longitudinal axis of the drill bit to be coaxial with a central longitudinal axis of the bushing.

For some applications, an inner diameter of the guide-sleeve is 3-6 mm.

For some applications, an outer diameter of the guide-sleeve is 3.5-6.5 mm.

For some applications, an inner diameter of the guide-sleeve is 3-6 mm, an outer diameter of the guide-sleeve is 3.5-6.5 mm, and the outer diameter is 0.5-1.5 mm larger than the inner diameter.

For some applications, the apparatus further includes the drill bit.

For some applications, an inner diameter of the guide-sleeve is within 0.2-3.5 mm of a diameter of a shank of the drill bit.

For some applications, when the guide-sleeve and drill bit are coupled to the distal end of the dental handpiece, a distance between a distal tip of the drill bit and the distal end of the guide-sleeve is between 5 mm and 25 mm.

For some applications, the guide-sleeve is configured to be immovable with respect to the drill bit when the guide-sleeve and drill bit are coupled to the distal end of the dental handpiece.

For some applications, the apparatus further includes the bushing.

For some applications, an outer diameter of the guide-sleeve is within 100 microns of an inner diameter of the bushing.

For some applications, the guide-sleeve is configured to prevent the drill bit from contacting the bushing when (a) the guide-sleeve and drill bit are coupled to the distal end of the dental handpiece, and (b) the guide-sleeve is in the bushing.

For some applications, the apparatus further includes the oral surgical guide.

For some applications, the apparatus further includes the dental handpiece.

For some applications, the dental handpiece is shaped to define an irrigation channel, and the guide-sleeve is shaped to define an opening to accommodate irrigation flow from the irrigation channel of the dental handpiece.

For some applications, the apparatus further includes at least one protrusion, protruding from the proximal end of the guide-sleeve, configured to lockingly engage a corresponding hole in the distal end of the dental handpiece.

For some applications, the apparatus further includes one or more protrusions, protruding from the proximal end of the guide-sleeve.

For some applications, the guide-sleeve is shaped to define an opening to accommodate irrigation flow from an irrigation channel of the dental handpiece.

For some applications, the apparatus further includes one or more protrusions, protruding from the proximal end of the guide-sleeve.

For some applications, the guide-sleeve is configured to prevent the drill bit from contacting the bushing when (a) the guide-sleeve and drill bit are coupled to the distal end of the dental handpiece, and (b) the guide-sleeve is in the bushing.

There is further provided, in accordance with some applications of the present invention, a method for aligning a drill bit with a bushing in an oral surgical guide, the method including:

placing a distal tip of the drill bit into the bushing such that there is an angle of 5-45 degrees between a central longitudinal axis of the drill bit and a central longitudinal axis of the bushing; and

subsequently aligning the central longitudinal axis of the drill bit to be coaxial with the central longitudinal axis of the bushing by aligning a central longitudinal axis of a rigid guide-sleeve with the central longitudinal axis of the bushing,
the guide-sleeve (a) being coupled to a distal end of a dental handpiece, (b) being sized and shaped to be slidably couplable with the bushing, and (c) having a central longitudinal axis that is coaxial with the central longitudinal axis of the drill bit.

For some applications, placing the distal tip of the drill bit into the bushing includes placing the distal end of the drill bit into the bushing such that there is 5-25 degrees between the central longitudinal axis of the drill bit and the central longitudinal axis of the bushing.

There is further provided, in accordance with some applications of the present invention, a method for assembling dental apparatus, the method including:

coupling a rigid guide-sleeve to a distal end of a dental handpiece; and

coupling a drill bit to the distal end of the dental handpiece such that a central longitudinal axis of the drill bit is coaxial with a central longitudinal axis of the guide-sleeve.

There is further provided, in accordance with some applications of the present invention, a method for aligning a drill bit with a bushing in an oral surgical guide, the method including:

placing a distal tip of the drill bit into the bushing;

using a rigid guide-sleeve that is coupled to a distal end of a dental handpiece to align a central longitudinal axis of the drill bit with a central longitudinal axis of the bushing, by:
(a) aligning a central longitudinal axis of the guide-sleeve with the central longitudinal axis of the bushing,
(b) the aligning of the central longitudinal axis of the guide-sleeve with the central longitudinal axis of the bushing reorienting the dental handpiece, and
(c) the reorienting by the guide-sleeve of the dental handpiece in turn reorienting the drill bit; and advancing the reoriented drill bit into tissue of a subject.

For some applications, placing the distal tip of the drill bit into the bushing includes placing the distal tip of the drill bit into the bushing such that there is an angle of 5-45 degrees between a central longitudinal axis of the drill bit and a central longitudinal axis of the bushing.

For some applications, placing the distal tip of the drill bit into the bushing includes placing the distal end of the drill bit into the bushing such that there is 5-25 degrees between the central longitudinal axis of the drill bit and the central longitudinal axis of the bushing.

There is further provided, in accordance with some applications of the present invention, apparatus for use with a dental handpiece, a drill bit, an oral surgical guide, and a bushing disposed in the oral surgical guide, the apparatus including:

a rigid guide-sleeve, the guide-sleeve being (a) removably couplable to a distal end of the dental handpiece, (b) configured to remain stationary during rotation of the drill bit when coupled to the dental handpiece, and (c) shaped to define an opening to accommodate flow from an irrigation channel disposed on the dental handpiece.

For some applications, the apparatus further includes at least one protrusion, protruding from a proximal end of the guide-sleeve, configured to lockingly engage a corresponding hole in a distal end of the dental handpiece.

The present invention will be more fully understood from the following detailed description of applications thereof, taken together with the drawings, in which:

### BRIEF DESCRIPTION OF THE DRAWINGS

FIG. 1 is an exploded schematic illustration of a guide-sleeve and a dental handpiece, in accordance with an application of the present invention;

FIG. 2 is a schematic illustration of different views of the guide-sleeve for use with a dental handpiece, drill bit, bushing, and oral surgical guide, in accordance with an application of the present invention;

FIG. 3 is a schematic illustration of the guide-sleeve coupled to the dental handpiece, and a drill bit, in accordance with an application of the present invention;

FIG. 4 is a schematic illustration of the guide-sleeve and drill bit, both coupled to the dental handpiece, in accordance with an application of the present invention;

FIG. 5 is a schematic illustration of an irrigation channel on the dental handpiece and an opening in the guide-sleeve to accommodate an irrigation flow, in accordance with an application of the present invention;

FIG. 6 is an exploded schematic illustration of the bushing and oral surgical guide suspended above an implant site, in accordance with an application of the present invention.

FIG. 7 is a schematic illustration of the bushing and oral surgical guide positioned on the implant site, in accordance with an application of the present invention;

FIG. 8 is a schematic illustration of the guide-sleeve coupled to the dental handpiece, where the guide-sleeve is fully inside the bushing, in accordance with an application of the present invention; and

FIG. 9 is a schematic illustration of movement of the guide-sleeve and drill bit, both coupled to the dental handpiece, where the distal tip of the drill bit is in the bushing, in accordance with an application of the present invention.

### DETAILED DESCRIPTION

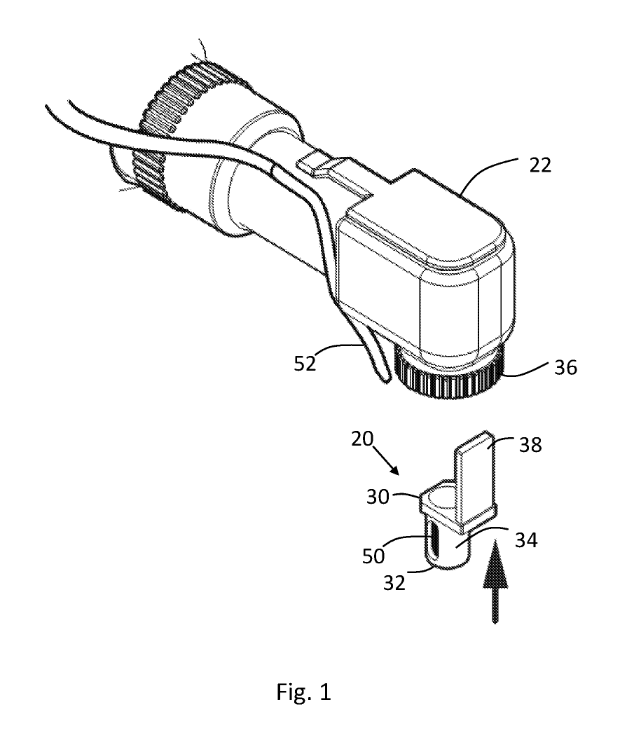

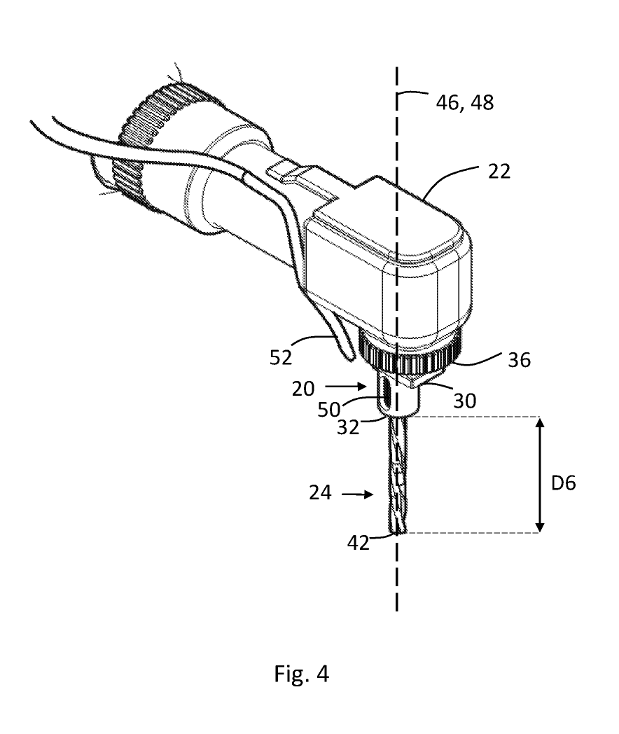

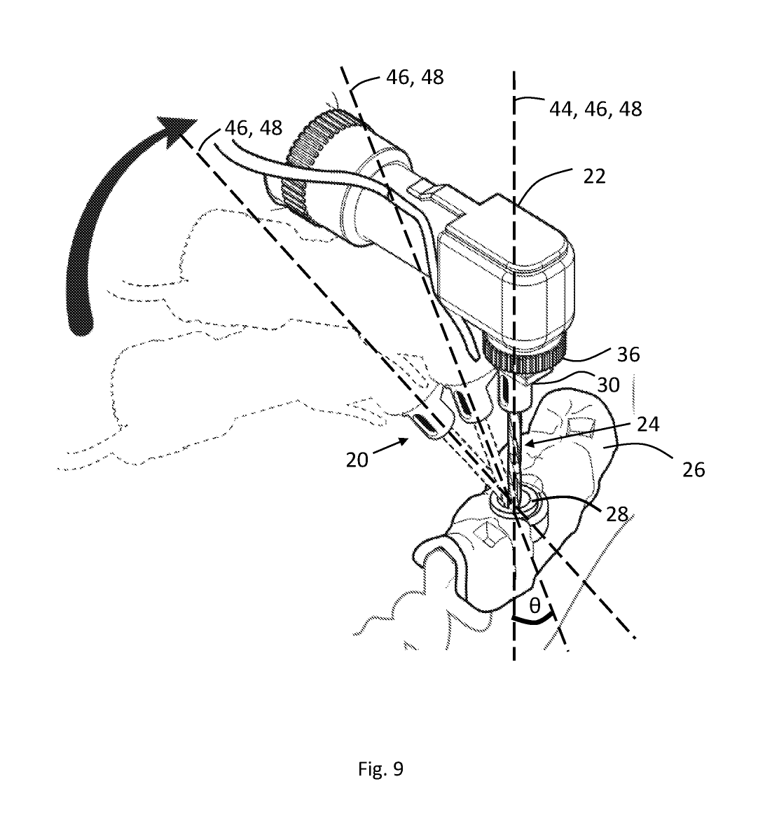

Reference is made to FIGS. 1 and 9. FIG. 1 is an exploded schematic illustration of a guide-sleeve 20 for use with a dental handpiece 22, in accordance with some applications of the present invention. FIG. 9 is a schematic illustration of guide-sleeve 20 being used with a drill bit 24, an oral surgical guide 26, and a bushing 28 disposed in oral surgical guide 26, in accordance with some applications of the present invention. Guide-sleeve 20 has a proximal end 30 and a distal end 32. Proximal end 30 of guide-sleeve 20 is removably couplable to a distal end 36 of dental handpiece 22, such that a distal outer surface 34 of guide-sleeve 20 and drill bit 24 (FIG. 4) coupled to distal end 36 of dental handpiece 22, are coaxial along a central longitudinal axis 46 of drill bit 24.

As used in the present application, including in the claims, a “dental handpiece” is to be understood as referring to the handpiece of a dental drilling instrument.

As used in the present application, including in the claims, a “central longitudinal axis” of an elongate structure is the set of all centroids of transverse cross-sectional sections of the structure along the structure. Thus, the cross-sectional sections are locally perpendicular to the central longitudinal axis, which runs along the structure. (If the structure is circular in cross-section, the centroids correspond with the centers of the circular cross-sectional sections.)

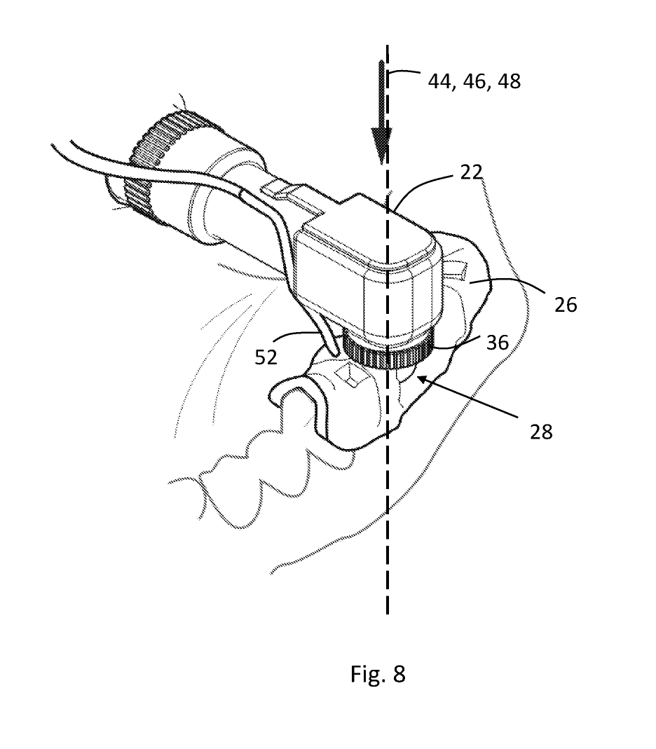

Distal end 32 of guide-sleeve 20 is sized and shaped to be slidably couplable with bushing 28, as shown in FIGS. 8 and 9, such that upon guide-sleeve 20 being advanced into bushing 28, dental handpiece 22 is forced to align central longitudinal axis 46 of drill bit 24 to be coaxial with a central longitudinal axis 44 of bushing 28.

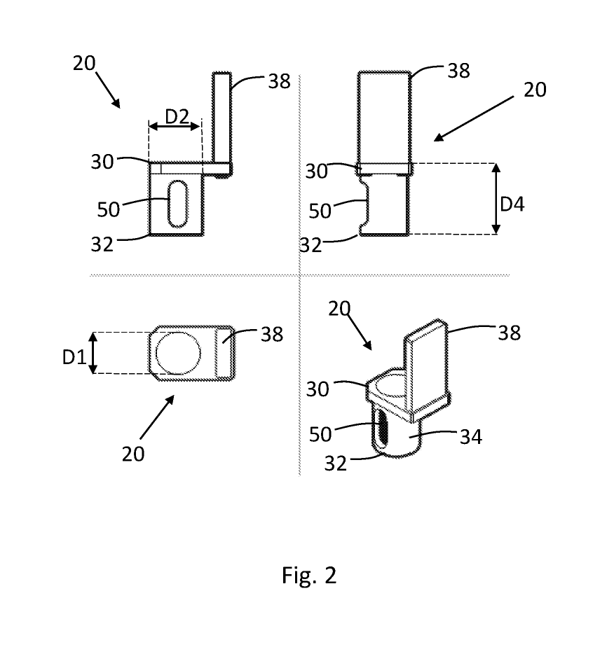

Reference is made to FIG. 2, which is a schematic illustration of guide-sleeve 20, in accordance with some applications of the present invention.

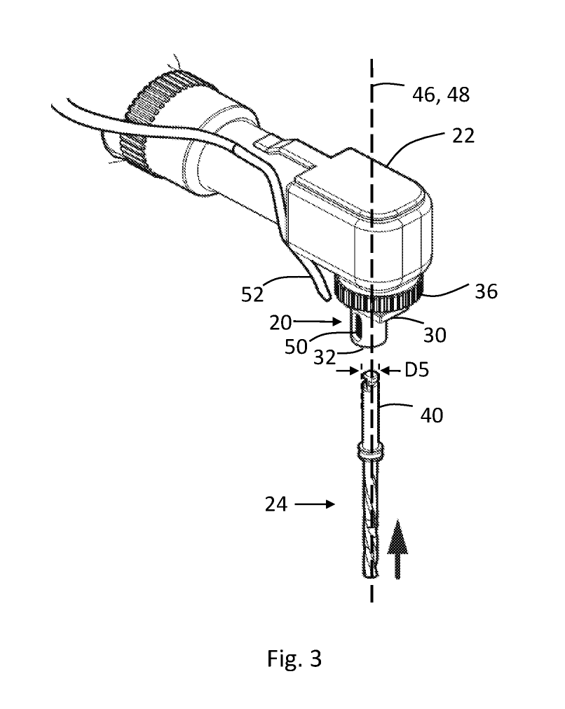

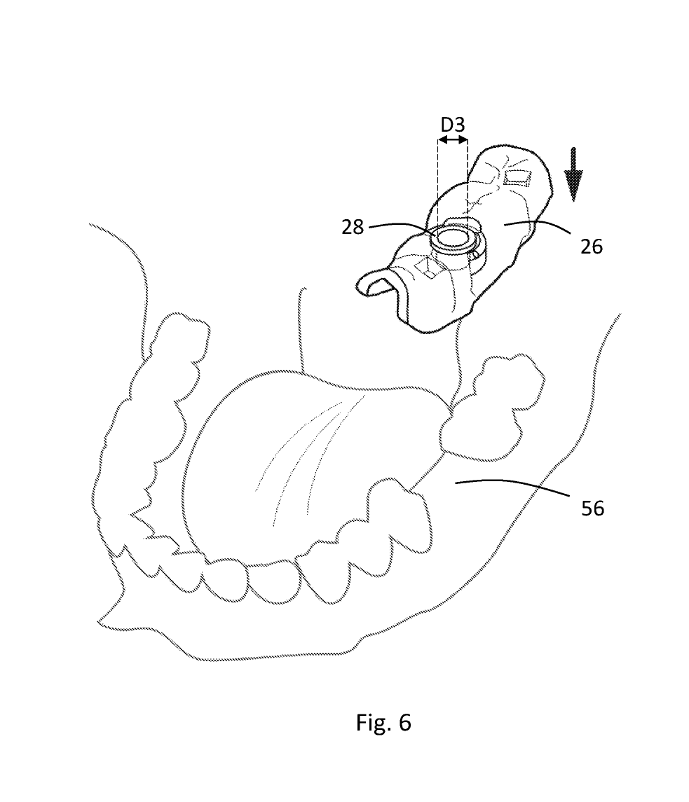

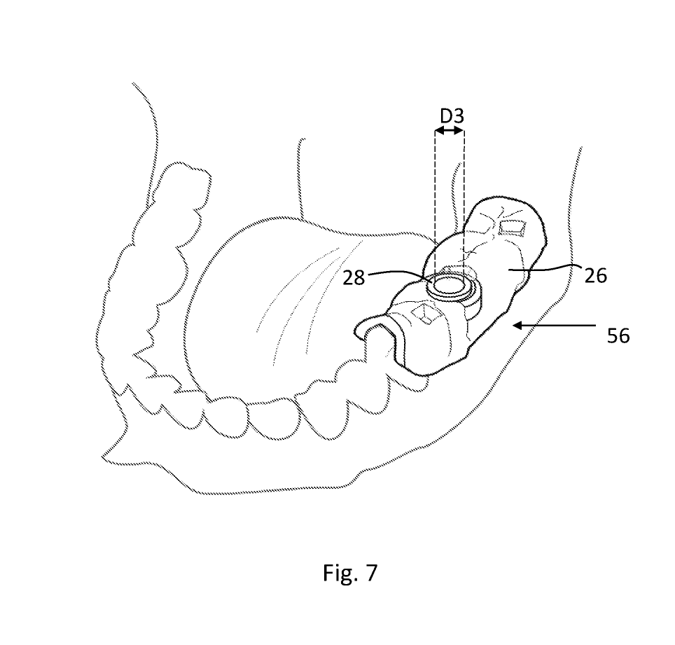

For some applications, the dimensions of guide sleeve 20 are set using one or more of the following options:
Guide-sleeve 20 is typically sized and shaped such that an inner diameter D1 of guide-sleeve 20 is at least 3 mm and/or less than 6 mm, an outer diameter D2 of guide-sleeve 20 is at least 3.5 mm and/or less than 6.5 mm, and outer diameter D2 of guide-sleeve 20 is 0.5-1.5 mm larger than inner diameter D1;
The height D4 of guide-sleeve 20 is typically at least 5 mm and/or less than 10 mm;
Inner diameter D1 of guide-sleeve 20 is typically within 0.2-3.5 mm of a diameter D5 of a shank 40 of drill bit 24, as shown in FIG. 3; and/or
Outer diameter D2 of guide-sleeve 20 is typically within 100 microns of an inner diameter D3 of bushing 28, as shown in FIGS. 6 and 7.

In an application of the present invention, guide-sleeve 20 comprises at least one protrusion 38, protruding from the proximal end 30 of guide-sleeve 20. Protrusion 38 is configured to lockingly engage a corresponding hole (not shown) in distal end 36 of dental handpiece 22. For some applications, guide-sleeve 20 comprises one or more protrusions 38 (configuration not shown), protruding from proximal end 30 of guide-sleeve 20.

Reference is now made to FIG. 3, which is a schematic illustration of the apparatus, in which guide-sleeve 20 is coupled to distal end 36 of dental handpiece 22, and drill bit 24 is shown suspended below distal end 36 of dental handpiece 22 so as to show how drill bit 24 is inserted into distal end 36 of dental handpiece 22. Guide-sleeve 20 and drill bit 24 are aligned such that a central longitudinal axis 48 of guide-sleeve 20 is coaxial with central longitudinal axis 46 of drill bit 24.

Reference is now made to FIG. 4, which is a schematic illustration of the apparatus in which guide-sleeve 20 and drill bit 24 are coupled to distal end 36 of dental handpiece 22. Guide-sleeve 20 is configured to be immovable with respect to drill bit 24 when guide-sleeve 20 and drill bit 24 are coupled to distal end 36 of dental handpiece 22. A distance D6 between a distal tip 42 of drill bit 24 and distal end 32 of guide-sleeve 20 is typically at least 5 mm and/or less than 25 mm when guide-sleeve 20 and drill bit 24 are coupled to distal end 36 of dental handpiece 22.

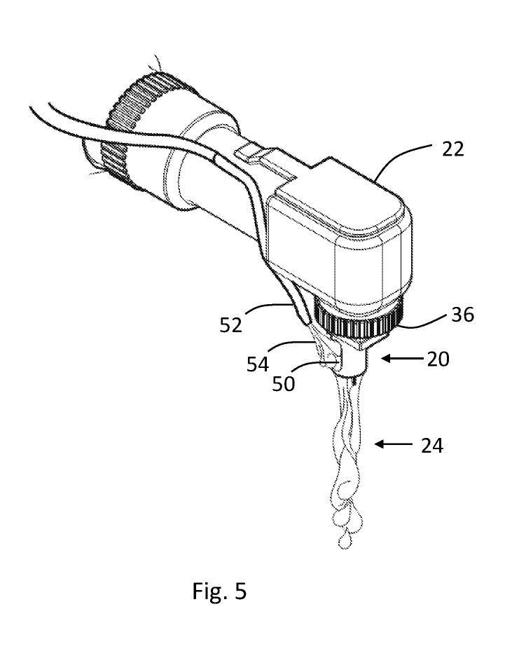

Reference is now made to FIG. 5, which is a schematic illustration of the apparatus, in which guide-sleeve 20 and drill bit 24 are shown to be coupled to distal end 36 of dental handpiece 22. Guide-sleeve 20 is shaped to define an opening 50 to accommodate an irrigation flow 54 from an irrigation channel 52 on dental handpiece 22. Typically, guide-sleeve 20 is configured to remain stationary during rotation of drill bit 24 when guide-sleeve 20 is coupled to distal end 36 of dental handpiece 22.

Reference is now made to FIGS. 6-7, which are schematic illustrations of bushing 28, disposed in oral surgical guide 26. Oral surgical guide 26 is shown suspended above an implant site 56 in FIG. 6, and positioned on implant site 56 in FIG. 7.

Reference is now made to FIG. 8, which is a schematic illustration of guide-sleeve 20 and drill bit 24 coupled to distal end 36 of dental handpiece 22, where guide-sleeve 20 is fully inside bushing 28, aligned such that a central longitudinal axis 48 of guide-sleeve 20 is coaxial with central longitudinal axis 44 of bushing 28. (Guide sleeve 20 and drill bit 24 are not visible, as they are inside oral surgical guide 26.) Guide-sleeve 20 prevents drill bit 24 from contacting bushing 28, when guide-sleeve 20 and drill bit 24 are coupled to distal end 36 of dental handpiece 22 and when guide-sleeve 20 is in bushing 28.

Reference is now made to FIG. 9, which is a schematic illustration of movement of guide-sleeve 20 and drill bit 24, both coupled to dental handpiece 22, where distal tip 42 of drill bit 24 is in bushing 28, in accordance with an application of the present invention. For some applications, a method is provided for aligning drill bit 24 with bushing 28 in oral surgical guide 26. The method comprises placing distal tip 42 of drill bit 24 into bushing 28 at an angle theta, such that the smaller of the two angles formed between central longitudinal axis 46 of drill bit 24 and central longitudinal axis 44 of bushing 28 is typically at least 5 degrees and/or less than 45 degrees. Subsequently, the dentist aligns central longitudinal axis 48 of guide-sleeve 20 to be coaxial with central longitudinal axis 44 of bushing 28, as shown in FIG. 3. Aligning central longitudinal axis 48 of guide-sleeve 20 with central longitudinal axis 44 of bushing 28 forces dental handpiece 22 to reorient, in turn forcing central longitudinal axis 46 of drill bit 24 to be coaxial with central longitudinal axis 44 of the bushing 28. Once drill bit 24 is aligned, it can be advanced into the tissue of the patient. In some applications, angle theta is typically at least 5 degrees and/or less than 25 degrees.

It is noted that by providing the dentist with the ability to initially place drill bit 24 in bushing 28 at angle theta allows the patient to open her mouth less wide, and thus reduces discomfort of the patient during the procedure and/or allows the performance of a procedure in a case where it would be difficult for the patient to open her mouth sufficiently wide.

For some applications, dental apparatus useful in procedures such as those described hereinabove is assembled by coupling guide-sleeve 20 to distal end 36 of dental handpiece 22, and coupling drill bit 24 to distal end 36 of dental handpiece 22, such that central longitudinal axis 46 of drill bit 24 is coaxial with central longitudinal axis 48 of guide-sleeve 20, as shown in FIG. 3 and FIG. 4.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for use with a dental handpiece, a drill bit, an oral surgical guide, and a bushing disposed in the oral surgical guide, the apparatus comprising:
a rigid guide-sleeve having a proximal end and a distal end,
the proximal end of the guide-sleeve being removably couplable to a distal end of the dental handpiece, such that a distal outer surface of the guide-sleeve and a drill bit coupled to the distal end of the dental handpiece are coaxial along a central longitudinal axis of the drill bit, and
the distal end of the guide-sleeve being sized and shaped to be slidably couplable with the bushing, such that upon the guide-sleeve being advanced into the bushing the dental handpiece is forced to align the central longitudinal axis of the drill bit to be coaxial with a central longitudinal axis of the bushing.

2. The apparatus according to claim 1, wherein an inner diameter of the guide-sleeve is 3-6 mm.

3. The apparatus according to claim 1, wherein an outer diameter of the guide-sleeve is 3.5-6.5 mm.

4. The apparatus according to claim 1, wherein an inner diameter of the guide-sleeve is 3-6 mm, an outer diameter of the guide-sleeve is 3.5-6.5 mm, and the outer diameter is 0.5-1.5 mm larger than the inner diameter.

5. The apparatus according to claim 1, further comprising the drill bit.

6. The apparatus according to claim 5, wherein an inner diameter of the guide-sleeve is within 0.2-3.5 mm of a diameter of a shank of the drill bit.

7. The apparatus according to claim 5, wherein when the guide-sleeve and drill bit are coupled to the distal end of the dental handpiece, a distance between a distal tip of the drill bit and the distal end of the guide-sleeve is between 5 mm and 25 mm.

8. The apparatus according to claim 5, wherein the guide-sleeve is configured to be immovable with respect to the drill bit when the guide-sleeve and drill bit are coupled to the distal end of the dental handpiece.

9. The apparatus according to claim 1, further comprising the bushing.

10. The apparatus according to claim 9, wherein an outer diameter of the guide-sleeve is within 100 microns of an inner diameter of the bushing.

11. The apparatus according to claim 9, wherein the guide-sleeve is configured to prevent the drill bit from contacting the bushing when (a) the guide-sleeve and drill bit are coupled to the distal end of the dental handpiece, and (b) the guide-sleeve is in the bushing.

12. The apparatus according to claim 9, further comprising the oral surgical guide.

13. The apparatus according to claim 1, further comprising the dental handpiece.

14. The apparatus according to claim 13, wherein the dental handpiece is shaped to define an irrigation channel, and wherein the guide-sleeve is shaped to define an opening to accommodate irrigation flow from the irrigation channel of the dental handpiece.

15. The apparatus according to claim 13, further comprising at least one protrusion, protruding from the proximal end of the guide-sleeve, configured to lockingly engage a corresponding hole in the distal end of the dental handpiece.

16. The apparatus according to claim 13, further comprising one or more protrusions, protruding from the proximal end of the guide-sleeve.

17. The apparatus according to claim 1, wherein the guide-sleeve is shaped to define an opening to accommodate irrigation flow from an irrigation channel of the dental handpiece.

18. The apparatus according to claim 1, further comprising one or more protrusions, protruding from the proximal end of the guide-sleeve.

19. The apparatus according to claim 1, wherein the guide-sleeve is configured to prevent the drill bit from contacting the bushing when (a) the guide-sleeve and drill bit are coupled to the distal end of the dental handpiece, and (b) the guide-sleeve is in the bushing.

20-27. (canceled)
